(19) United States
(12) Patent Application Publication
(10) Pub. No.: US 2026/0272403 A1
(43) Pub. Date: Sep. 17, 2026

(54) PERSISTENT DISPLAY OF BIOPSY TARGETS

(71) Applicant: Veran Medical Technologies, Inc., St. Louis, MO (US)

(72) Inventor: Christopher Lee, St. Louis, MO (US)

(21) Appl. No.: 19/161,875

(22) PCT Filed: Mar. 5, 2024
(86) PCT No.: PCT/US2024/018520

§ 371 Date: Sep. 3, 2025

Related U.S. Application Data
(60) Provisional application No. 63/489,392, filed on Mar. 9, 2023.

(51) Int. Cl. A61B 8/08; A61B 8/00; A61B 8/12; A61B 10/04; A61B 34/10

(52) U.S. Cl. CPC A61B 8/0841; A61B 8/085; A61B 8/12; A61B 8/463; A61B 8/5246; A61B 10/04; A61B 34/10; A61B 2010/045; A61B 2034/107

(57) ABSTRACT

Various aspects of methods, systems, and use cases may be used to generate and display a real-time a composite image, such as in a manner that combines a first US image in which a deep target is within the field of view (FOV) of an EBUS device together with one or more subsequently acquired second US images in which the deep target is no longer within the FOV of the EBUS device. In this way, the EBUS system persistently displays imaged biopsy targets even after the biopsy targets are no longer within the FOV of the US transducer.

### PRIORITY CLAIM

This application claims the benefit of priority to U.S. Provisional Patent Application Ser. No. 63/489,392, filed Mar. 9, 2023, the contents of which are incorporated herein by reference in their entirety.

### BACKGROUND

A solitary pulmonary nodule (SPN) is an isolated mass in the lung that is smaller than three centimeters in diameter and is surrounded by normal tissue. SPNs can be identified through common medical imaging techniques such as computerized tomography (CT) scans and positron emission tomography (PET) scans. In most cases SPNs and other lung nodules are merely benign tumors. In other cases, these SPNs are malignant cancers which must be treated to prevent premature mortality.

Diagnosis of identified SPNs cannot be performed via medical imaging alone but instead requires a biopsy which may be performed via an endobronchial ultrasound (EBUS) equipped with real-time sampling functionality. Real-time sampling with an EBUS device typically includes navigating the EBUS device through a patient's airways to a target nodule that was previously identified in a medical image so that the target nodule is visible in a real-time ultrasound (US) image. Then, while the target nodule is viewed in the US image, a sampling needle is extended from a working channel of the EBUS device into the target nodule to obtain a sample thereof.

### Overview

Modern EBUS devices include a ramp through which a biopsy needle may be extended into a field of view (FOV) of a US transducer. In particular, the ramp is located proximally from the US transducer and directs the biopsy needle into the FOV along a needle path axis that diverges away from the longitudinal axis of the EBUS device at an acute angle. Depending on the depth that an SPN resides in relation to an airway from which the biopsy is be collected, it may not be possible to collect a sample while the SPN remains within the FOV of the US transducer. In particular, the EBUS device may need to be moved proximal from where the SPN resides within the FOV in order for the needle path axis to converge with the SPN. To address these challenges the inventors have developed real-time US imaging techniques that mitigate the above challenges. The following drawings and specification text describe examples of these imaging techniques which facilitate persistent display of SPNs that fall outside the real-time FOV of the US transducer.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a schematic diagram of an EBUS targeting a nodule at a first distance in accordance with at least one example of this disclosure.

FIG. 2 illustrates a schematic diagram of tracking of the sampling needle of the EBUS of FIG. 1 targeting the nodule at the first distance in accordance with at least one example of this disclosure.

FIG. 3A illustrates a schematic diagram of the EBUS of FIG. 1 targeting a nodule at a second distance in accordance with at least one example of this disclosure.

FIG. 3B illustrates a schematic diagram of the EBUS of FIG. 3A targeting the nodule at the second distance in accordance with at least one example of this disclosure.

FIG. 4 illustrates a schematic diagram of tracking of the sampling needle of the EBUS of FIG. 3A targeting the nodule at the second distance in accordance with at least one example of this disclosure.

FIG. 5A illustrates a schematic diagram of tracking of the sampling needle of the EBUS of FIG. 3A in a first position and the nodule at the second distance in accordance with at least one example of this disclosure.

FIG. 5B illustrates a schematic diagram of tracking of the sampling needle of the EBUS of FIG. 3A in a second position and the nodule at the second distance in accordance with at least one example of this disclosure.

FIG. 5C illustrates a schematic diagram of tracking of the sampling needle of the EBUS of FIG. 3A in a third position and the nodule at the second distance in accordance with at least one example of this disclosure.

FIG. 5D illustrates a schematic diagram of tracking of the sampling needle of the EBUS of FIG. 3A in a fourth position intersecting the nodule at the second distance in accordance with at least one example of this disclosure.

FIG. 6 illustrates an example of a method of displaying a nodule when the nodule is no longer within the field of view of an EBUS without navigation in accordance with at least one example of this disclosure.

FIG. 7 illustrates an example of a method of displaying a nodule when the nodule is no longer within the field of view of an EBUS with navigation in accordance with at least one example of this disclosure.

FIG. 8 illustrates a block diagram of an example machine upon which any one or more of the techniques discussed herein may perform.

### DETAILED DESCRIPTION

The present disclosure is directed to combining a first US image in which a deep target is within the field of view (FOV) of an EBUS device together with one or more subsequently acquired second US images in which the deep target is no longer within the FOV of the EBUS device (e.g., due to the EBUS device having been moved proximally away from the target). In this way, the EBUS system persistently displays imaged biopsy targets even after the biopsy targets are no longer within the FOV of the US transducer. As described in more detail below, such techniques provide advantages over existing EBUS sampling systems by persistently displaying biopsy targets that are distal from the US FOV due to the EBUS sampling device being positioned proximally from the biopsy target to align a needle trajectory with the biopsy target.

In an example, the EBUS is a non-navigated EBUS (i.e., lacks a 6DoF sensor or other surgical navigation technology), and the combining of the images together may be continually monitored and updated to provide an estimate of the real time location of the target in relation to a needle path axis. Since the distal tip of the needle will be outside of the FOV when it pierces the target, in some examples, the EBUS may track a needle extension length and generate a virtual representation of the needle and/or needle tip on the displayed and stitched together US images. The needle extension length may be determined based data collected from a linear encoder in the needle actuation handle or any other suitable technique for measuring sampling needle extension.

In an example, the EBUS is a navigated EBUS, and the location of the target with respect to a current FOV may be calculated even after the target is no longer within the current FOV based on a comparison of first navigation data that is collected when the target is within the FOV to second navigation data collected after the target is no longer within the FOV (and potentially when the needle path axis converges with the target). The first navigation data indicates a first device pose (i.e., position and orientation) and the second navigation data indicates a second device pose. In this example, the combination of the second navigation data and the first navigation data may be used to determine a placement of a previously acquired ultrasound image (e.g., in which the target is within the FOV) with respect to a real-time image stream being captured by the EBUS. In this way, the previously acquired ultrasound image may be appropriately displayed in real time adjacent to the real-time image stream at a location with accurately indicates the target location relative to the real-time image stream. In this way, multiple images can be combined (e.g., stitched together) based on one or more identified locations of the SPN to provide a real-time display that includes past imaging of the SPN. In an example, the system can generate overlays including needle trajectory and/or estimated needle position.

Determining a current location of a targeted tissue (e.g., an SPN) with respect to a current US FOV after the targeted tissue is no longer within the current US FOV can be done based on image combining, based on navigation data, or a combination of these.

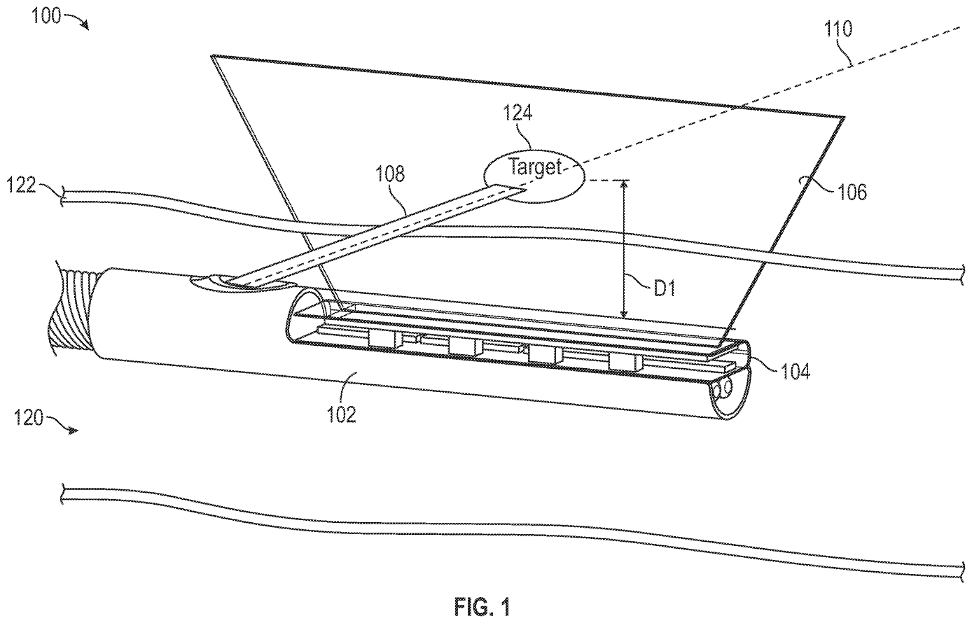

FIG. 1 illustrates a schematic diagram 100 of an EBUS 102 (e.g., a EBUS sampling device) targeting a nodule 124 at a first distance (D1) in accordance with at least one example of this disclosure. In the illustrated example, the EBUS 102 is positioned in an airway channel 120. The airway channel 120 is defined with airway tissue walls 122. The nodule 124 is located within the airway tissue walls 122. In an example, the EBUS is designed for single use.

The EBUS 102 includes an imaging sensor 104. In this example, the imaging sensor 104 is an ultrasound transducer such as, for example, a Piezoelectric Micromachined Ultrasonic Transducers (PMUT). In an example, the imaging sensor 104 is positioned at a distal end of the EBUS 102. In an example, the imaging sensor 104 extends along a portion of the distal end of the EBUS 102. In an example, the imaging sensor 104 has a substantially planar surface on which a plurality of ultrasound elements are disposed, and which is substantially orthogonal to a longitudinal axis of the EBUS device 102. In another example, the imaging sensor 104 is curved away from the EBUS 102. The imaging sensor 104 captures medical images within a field of view (FOV) 106. The FOV 106 can be transmitted to an imaging display.

The EBUS 102 includes a sampling needle 108. In an example, the EBUS 102 also includes a sheath surrounding a portion of the sampling needle 108. For example, the sheath is partially extended out of the EBUS 102 prior to the sampling needle 108 being extended from the EBUS 102. Such a configuration can protect an exit port of the EBUS 102 where the sampling needle 108 extends from the EBUS 102.

The sampling needle 108 extends out of the EBUS 102 at a sampling needle trajectory 110. In the illustrated example, the sampling needle trajectory 110 is predictable based on the angle that the sampling needle 108 exits the exit port of the EBUS 102 and a known length of the sampling needle 108. In the illustrated example, the sampling needle 108 and the sampling needle trajectory 110 pass through the nodule 124.

For example, the EBUS 102 is used to navigate towards and image the nodule 124 prior to and during sampling of the nodule 124. In an example, the nodule 124 is a preidentified tissue for which a biopsy sample is desired. In an example, ultrasound imaging, using the imaging sensor 104, is used in the respiratory area (e.g., airway channel 120 and airway tissue walls 122) to positively identify a target area from which to acquire a biopsy sample of the nodule 124 because the ultrasound imaging shows both tissue and an instrument (e.g., sample needle 108 and/or a sheath) in real time.

The FOV 106 extends from the airway channel 120 into the airway tissue walls 122. The FOV 106 may angle outward slightly from the edges of the imaging sensor 104, depending on a curvature of the imaging sensor 104. In the illustrated example, the nodule 124 is located at the first distance (D1) from the EBUS. As such, the FOV 106 is sufficiently sized to capture the nodule 124 at the first distance (D1). In addition, because the first distance (D1) of the nodule 124 is relatively shallow in the airway tissue walls 122, the EBUS device 102 may be positioned such that the sampling needle trajectory 110 intersects the target nodule 124 within the FOV 106. Therefore, the sampling needle 108 may be used to perform real-time visualization of the target nodule 124 while the needle trajectory 110 intersects the target 124 within the FOV 106. Therefore, a clinician can collect a biopsy sample from the nodule 124 while the nodule 124 and sampling needle 108 are being imaged in real time.

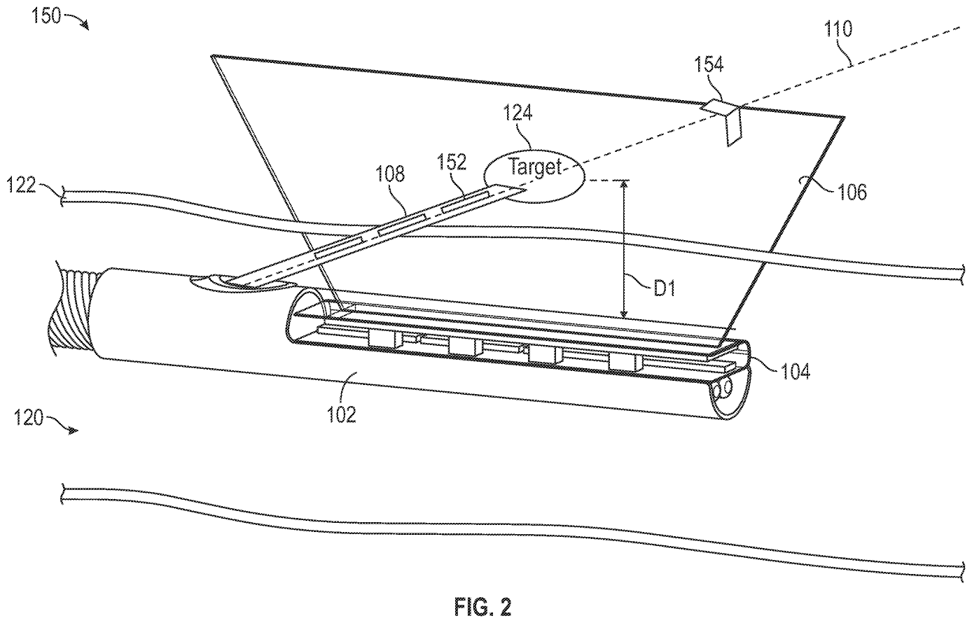

FIG. 2 illustrates a schematic diagram 150 of tracking of the sampling needle 108 of the EBUS 102 of FIG. 1 targeting the nodule 124 at the first distance (D1) in accordance with at least one example of this disclosure. In the illustrated example, the EBUS 102 is positioned in an airway channel 120. The airway channel 120 is defined with airway tissue walls 122. The nodule 124 is located within the airway tissue walls 122. The EBUS 102 is substantially similar, or the same as, EBUS 102 of FIG. 1. For example, the EBUS 102 of FIG. 2 may include an imaging sensor 104 and an exit port including a ramp that directs a sampling needle 108 along a sampling needle trajectory 110 into the FOV 106. The imaging sensor 104 captures the FOV 106. In an example, the imaging sensor 104 and the FOV 106 are substantially similar to the imaging sensor 104 and FOV 106 of FIG. 1.

The FOV 106 is transmitted and displayed on an imaging display. In the illustrated example, to aid the clinician in collecting the sample of the nodule 124 with the sampling needle 108, a sampling needle representation 152 is generated on the imaging display. For example, the sampling needle representation 152 is a graphical representation of the extension of the sampling needle 108. In an example, the sampling needle representation 152 dynamically changes as the sampling needle 108 continues to be extended or retracted in the FOV 106. In an example, the sampling needle representation 152 looks substantially similar to the sampling needle 108 to aid in identifying the sampling needle 108.

In the illustrated example, to aid the clinician in collecting the sample of the nodule 124 with the sampling needle 108, a sampling needle trajectory representation 154 is generated on the imaging display. For example, the sampling needle trajectory representation 152 is a graphical representation of the predicted trajectory of the sampling needle 108 (e.g., sampling needle trajectory 110). In an example, the sampling needle trajectory representation 154 can be an arrow (as shown), a line, or other representation of the sampling needle trajectory 110. In an example, the sampling needle trajectory representation 154 is positioned at an edge of the FOV 106. For example, a top edge or a distal edge of the FOV 106 based on the angle of the sampling needle trajectory 110. In another example, the sampling needle trajectory representation 154 is positioned a predetermined distance from the sampling needle representation 152. For example, as the sampling needle 108, and therefore the sampling needle representation 152, is extended or retracted, the sampling needle trajectory representation 154 moves along the sampling needle trajectory 110 at a fixed distance from the sampling needle representation 152. In some examples, multiple sampling needle trajectory representations 154 are generated on the imaging display.

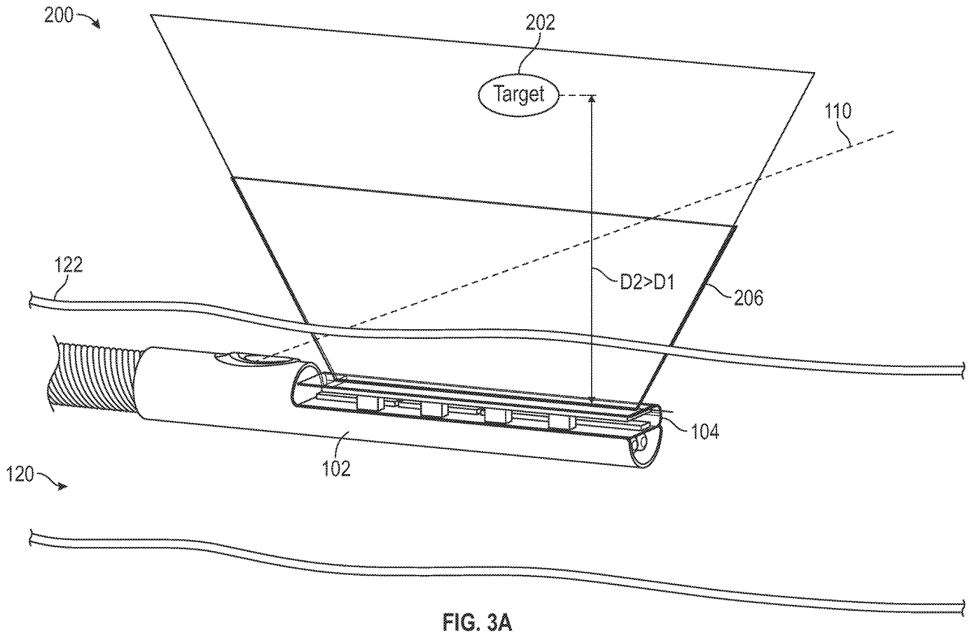

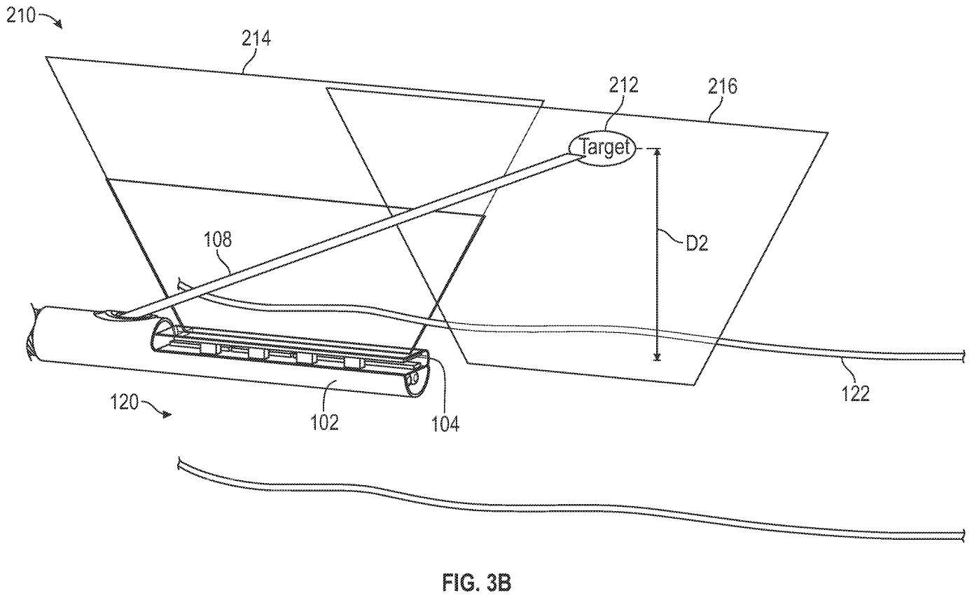

FIG. 3A illustrates a schematic diagram 200 of the EBUS 102 of FIG. 1 targeting a nodule 202 at a second distance (D2) in accordance with at least one example of this disclosure. In the illustrated example, the EBUS 102 is positioned in an airway channel 120. The airway channel 120 is defined with airway tissue walls 122. The nodule 202 is located within the airway tissue walls 122. The EBUS 102 is substantially similar, or the same as, EBUS 102 of FIG. 1. The EBUS 102 is substantially similar, or the same as, EBUS 102 of FIG. 1. For example, the EBUS 102 of FIG. 3A may include an imaging sensor 104 and an exit port including a ramp that directs a sampling needle 108 along a sampling needle trajectory 110 into a FOV 206. The imaging sensor 104 captures the FOV 206. In an example, the imaging sensor 104 is substantially similar to the imaging sensor of FIG. 1. In the illustrated example, the sampling needle trajectory 110 does not pass through the nodule 202.

For example, the EBUS 102 is used to navigate towards and image the nodule 202 prior to sampling of the nodule 202. In an example, the nodule 202 is a preidentified tissue for which a biopsy sample is desired. In an example, ultrasound imaging, using the imaging sensor 104, is used in the respiratory area (e.g., airway channel 120 and airway tissue walls 122) to positively identify a target area from which to acquire a biopsy sample of the nodule 202 because the ultrasound imaging shows both tissue and an instrument (e.g., sample needle and/or a sheath) in real time.

The FOV 206 extends from the airway channel 120 into the airway tissue walls 122. The FOV 206 may angle outward slightly from the edges of the imaging sensor 104, depending on a curvature of the imaging sensor 104. In the illustrated example, the nodule 202 is located at the second distance (D2) from the EBUS. The second distance (D2) is greater than the first distance (D1) of FIG. 1. As such, the FOV 206 is sufficiently sized to capture the nodule 202 at the second distance (D2). However, because the second distance (D2) of the nodule 202 is relatively deep in the airway tissue walls 122, while both the nodule 202 and the sampling needle trajectory 110, and therefore the sampling needle, are simultaneously within the FOV 206, the sampling needle trajectory 110 does not pass through the nodule 202 within the FOV 206 while being imaged in real time. In particular, as illustrated, the sampling needle trajectory 110 begins at the side exit ramp of the EBUS device 102 and diverges away from the longitudinal axis of the EBUS device 102. Due to the sample needle trajectory 110 diverging away from the longitudinal axis while traversing from the proximal boundary of the FOV 206 to the distal boundary of the FOV 206, the needle trajectory 110 passes through the distal boundary of the FOV 206 at a particular depth. It will be appreciated that target nodules which are deeper within the tissue (e.g., farther from the airway wall) than the depth at which the needle trajectory passes through the distal boundary of the FOV cannot be simultaneously sampled with a needle following the needle trajectory 110 while also being imaged by the imaging sensor 104.

FIG. 3B illustrates a schematic diagram 210 of the EBUS 102 of FIG. 3A targeting the nodule 212 at the second distance (D2) in accordance with at least one example of this disclosure. In the illustrated example, the EBUS 102 is positioned in an airway channel 120. The airway channel 120 is defined with airway tissue walls 122. The nodule 212 is located within the airway tissue walls 122. The EBUS 102 is substantially similar, or the same as, EBUS 102 of FIG. 1 and FIG. 2A.

In the illustrated example, the imaging sensor 104 captures a first (previous) field of view (FOV) 216. The previous FOV 216 may be captured at a moment in time when the needle trajectory does not intersect with the target nodule 212 and when the target nodule 212 is within the FOV of the imaging sensor 104 (e.g., when the EBUS device 102 is positioned as shown in FIG. 3A). The previous FOV 216 can be transmitted to an imaging display. The imaging sensor 104 is then translated through the airway channel 120 to capture a second (current) FOV 214. The current FOV 214 may be captured at a moment in time when the needle trajectory does intersect the target nodule 212 but when the target nodule 212 is no longer within the FOV of the imaging sensor 104 (e.g., when the EBUS device 102 is positioned as shown in FIG. 3B). The current FOV 214 can be transmitted to an imaging display in real time. The current FOV 214 and the previous FOV 216 extends from the airway channel 120 into the airway tissue walls 122. The current FOV 214 and the previous FOV 216 may angle outward slightly from the edges of the imaging sensor 104, depending on a curvature of the imaging sensor 104. In the illustrated example, the nodule 212 is located at the second distance (D2) from the EBUS. The second distance (D2) is greater than the first distance (D1) of FIG. 1. As such, the current FOV 214 and the previous FOV 216 is sufficiently sized to capture the nodule 212 at the second distance (D2). In the illustrated example, the sampling needle 108 passes through the nodule 212. However, because the second distance (D2) of the nodule 212 is relatively deep in the airway tissue walls 122, the sampling needle 108 does not pass through the nodule 212 within the current FOV 214 while being imaged in real time. Instead, the current FOV 214 captures the sampling needle 108 being extended from the EBUS 102 and the previous FOV 216 had captured the nodule 212.

In this example, the current FOV 214 and the previous FOV 216 can be stitched together to generate a composite FOV. The composite FOV can then be displayed with a real-time image of the sampling needle 108 within the current FOV 214 portion of the composite FOV. Once the sampling needle 108 extends into the previous FOV 216 portion of the composite FOV, then a graphical representation of the predicted needle location can be overlaid on the composite FOV to assist the clinician in targeting the nodule 212.

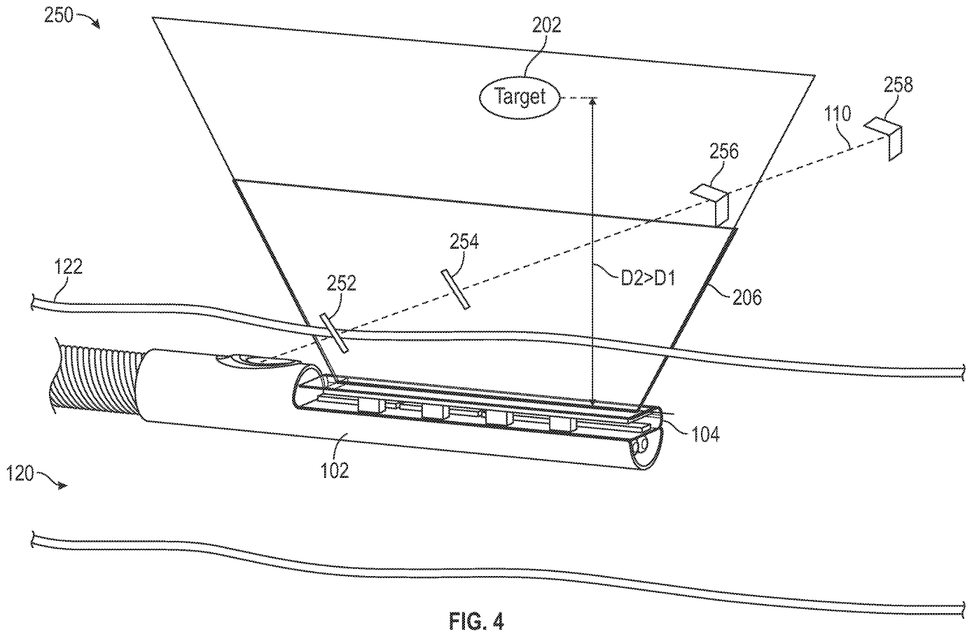

FIG. 4 illustrates a schematic diagram 250 of tracking of the sampling needle of the EBUS 102 of FIG. 3A targeting the nodule 202 at the second distance (D2) in accordance with at least one example of this disclosure. In the illustrated example, the EBUS 102 is positioned in an airway channel 120. The airway channel 120 is defined with airway tissue walls 122. The nodule 202 is located within the airway tissue walls 122. The EBUS 102 is substantially similar, or the same as, EBUS 102 of FIG. 1. For example, the EBUS 102 of FIG. 4 may include an imaging sensor 104 and an exit port including a ramp that directs a sampling needle 108 along a sampling needle trajectory 110 into a FOV 206. The imaging sensor 104 captures the FOV 206. In an example, the imaging sensor 104 is substantially similar to the imaging sensor 104 of FIG. 1. The FOV 206 can be transmitted to an imaging display. The FOV 206 may be substantially similar the FOV 206 of FIG. 3A.

The EBUS 102 includes a sampling needle. In an example, the EBUS 102 also includes a sheath surrounding a portion of the sampling needle. For example, the sheath is partially extended out of the EBUS 102 prior to the sampling needle being extended from the EBUS 102. Such a configuration can protect an exit port of the EBUS 102 where the sampling needle extends from the EBUS 102. In the illustrated example, the sampling needle trajectory 110 does not pass through the nodule 202 in the FOV 206.

For example, the EBUS 102 is used to navigate towards and image the nodule 202 prior to sampling of the nodule 202. In an example, the nodule 202 is a preidentified tissue for which a biopsy sample is desired. In an example, ultrasound imaging, using the imaging sensor 104, is used in the respiratory area (e.g., airway channel 120 and airway tissue walls 122) to positively identify a target area from which to acquire a biopsy sample of the nodule 202 because the ultrasound imaging shows both tissue and an instrument (e.g., sample needle and/or a sheath) in real time.

The FOV 206 is transmitted and displayed on an imaging display. In the illustrated example, to aid the clinician in collecting the sample of the nodule 202 with the sampling needle, a sampling needle representation 252 is generated on the imaging display. For example, the sampling needle representation 252 is a graphical representation of the extension of the sampling needle. In an example, the sampling needle representation 252 dynamically changes as the sampling needle continues to be extended or retracted in the FOV 206. In an example, the sampling needle representation 252 is an indication perpendicular to the sampling needle trajectory 110. In another example, the sampling needle representation 252 looks substantially similar to the sampling needle to aid in identifying the sampling needle. In another example, multiple sampling needle representations 252 and 254 are generated. For example, a first sampling needle representation 252 identifies that the sampling needle is extended and a second sampling needle representation 254 indicates a distance that the sampling needle is extended.

In the illustrated example, to aid the clinician in collecting the sample of the nodule 202 with the sampling needle, a sampling needle trajectory representation 256 is generated on the imaging display. For example, the sampling needle trajectory representation 256 is a graphical representation of the predicted trajectory of the sampling needle (e.g., sampling needle trajectory 110). In an example, the sampling needle trajectory representation 256 can be an arrow (as shown), a line, or other representation of the sampling needle trajectory 110. In an example, the sampling needle trajectory representation 256 is positioned at an edge of the FOV 206. For example, a top edge or a distal edge of the FOV 206 based on the angle of the sampling needle trajectory 110. In another example, the sampling needle trajectory representation 256 is positioned a predetermined distance from the sampling needle representation 252 or 254. For example, as the sampling needle, and therefore the sampling needle representation 252 or 254, is extended or retracted, the sampling needle trajectory representation 256 moves along the sampling needle trajectory 110 at a fixed distance from the sampling needle representation 252 or 254. In some examples, multiple sampling needle trajectory representations 256 and 258 are generated on the imaging display.

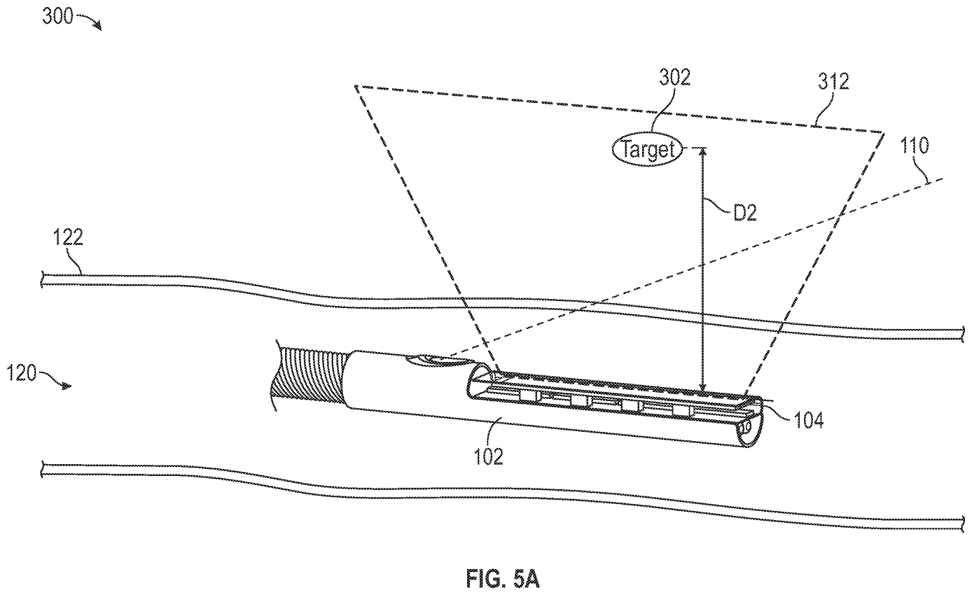

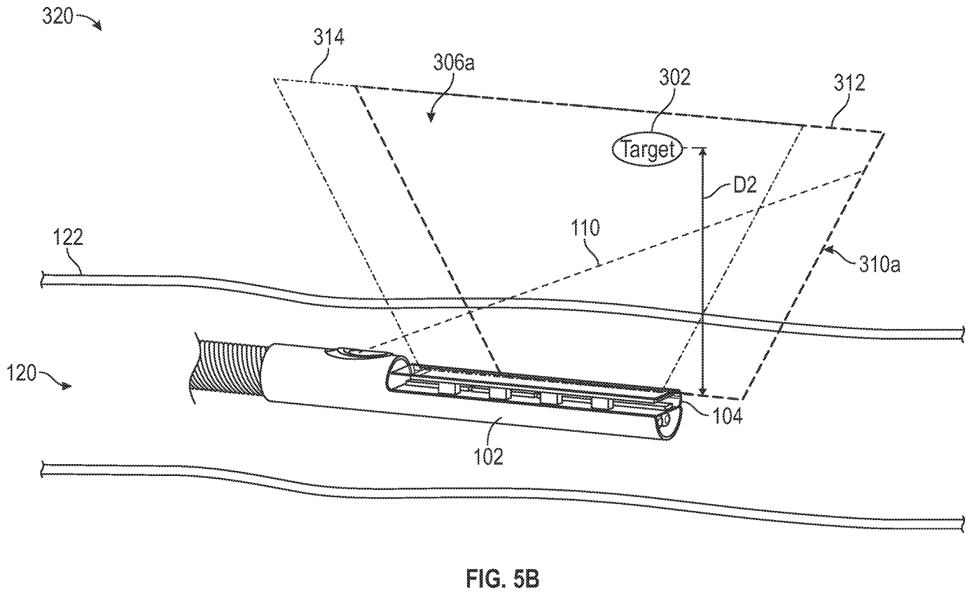

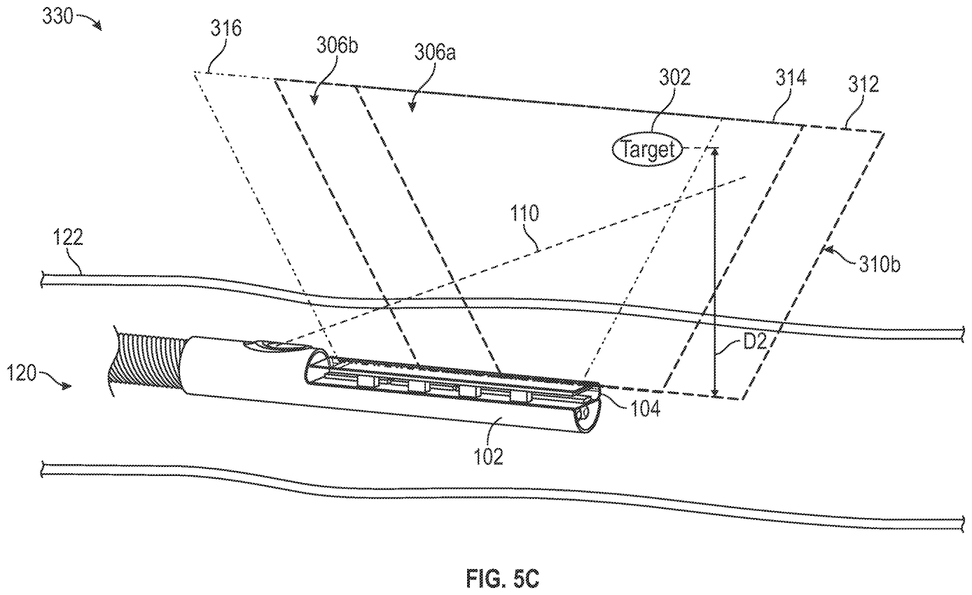

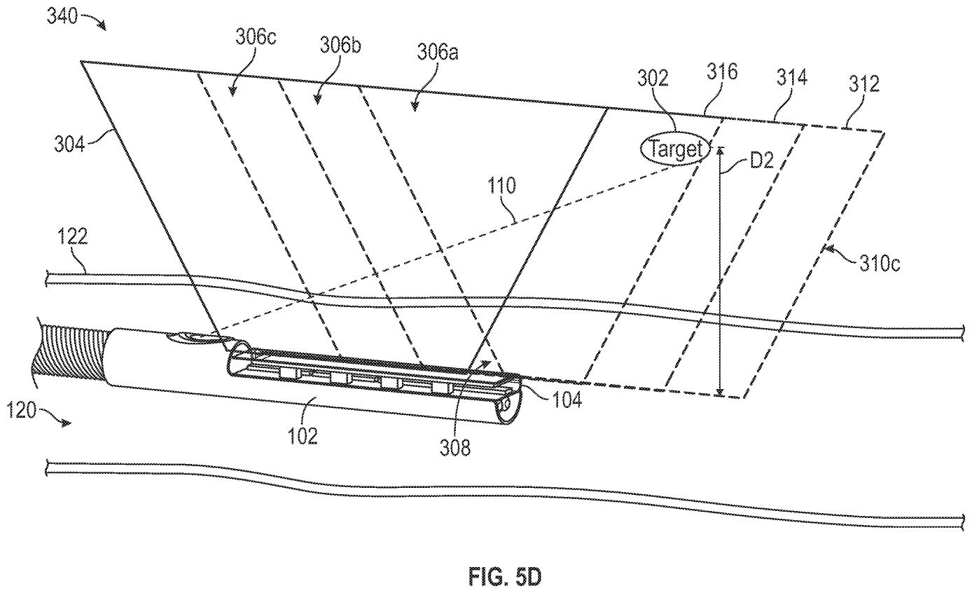

FIGS. 5A-5D illustrate schematic diagrams of tracking of the sampling needle 108 of the EBUS 102 of FIG. 3A at various positions 300, 320, 330, and 340 and a nodule 302 at the second distance (D2) in accordance with at least one example of this disclosure.

In the illustrated example, the EBUS 102 is positioned in an airway channel 120. The airway channel 120 is defined with airway tissue walls 122. The nodule 302 is located within the airway tissue walls 122. The EBUS 102 is substantially similar, or the same as, EBUS 102 of FIG. 1. For example, the EBUS 102 of FIGS. 5A-5D may include an imaging sensor 104 and an exit port including a ramp that directs a sampling needle along a sampling needle trajectory 110 into a FOV. The imaging sensor 104 captures a FOV. In an example, the imaging sensor 104 and is substantially similar to the imaging sensor 104 of FIG. 1.

The FOV extends from the airway channel 120 into the airway tissue walls 122. The FOV may angle outward slightly from the edges of the imaging sensor 104, depending on a curvature of the imaging sensor 104. In the illustrated example, the nodule 302 is located at the second distance (D2) from the EBUS 102. The second distance (D2) is greater than the first distance (D1) of FIG. 1. However, because the second distance (D2) of the nodule 302 is relatively deep in the airway tissue walls 122, both the nodule 302 and the sampling needle are not simultaneously within a current FOV 304 while being imaged in real time.

In the illustrated example, the EBUS 102 starts in a first, distal most position 300 (see FIG. 5A) to capture a first FOV 312. The first FOV 312 is transmitted to the imaging display. The first FOV 312 can include a representation of the sampling needle trajectory 110. In the first position 300, the sampling needle trajectory 110 does not intersect the nodule 302. With the EBUS 102 in the first position 300, the nodule 302 is captured in the first FOV 312.

As the EBUS 102 is withdrawn proximally in the airway channel 120, the EBUS 102 is moved to a second position 320 (see FIG. 5B) to capture a second FOV 314. The second FOV 314 is transmitted to the imaging display. In the illustrated example, the second FOV 314 and the first FOV 312 are combined to generate a composite image 310a. The composite image 310a is displayed on the imaging device. In the illustrated example, the first FOV 312 and the second FOV 314 have an overlapping portion 306a. Because there is an overlapping portion 306a, the more recently obtained FOV, here the second FOV 314, is displayed for the overlapping portion 306a. Therefore, only a portion of the first FOV 312 is used for the composite image 310a. The composite image 310a can include a representation of the sampling needle trajectory 110. In the second position 320, the sampling needle trajectory 110 does not intersect the nodule 302.

As the EBUS 102 is further withdrawn proximally in the airway channel 120, the EBUS 102 is moved to a third position 330 (see FIG. 5C) to capture a third FOV 316. The third FOV 316 is transmitted to the imaging display. In the illustrated example, the third FOV 316, the second FOV 314, and the first FOV 312 are combined to generate a composite image 310b. In another example, the third FOV 316 and the second FOV 314 are combined to generate a composite image. For example, the images are combined only as distally as needed to maintain the nodule 302 (e.g., a portion of the nodule 302, a substantial portion of the nodule 302, or the entire nodule 302) within the composite image. The composite image 310b is displayed on the imaging device. In the illustrated example, the first FOV 312 and the second FOV 314 have an overlapping portion 306a, and the second FOV 314 and the third FOV 316 have an overlapping portion 306b. A portion of the overlapping portion 306a is an overlap between the first FOV 312 and the third FOV 316. Because there is an overlapping portion 306a, and an overlapping portion 306b, the more recently obtained FOV, here the second FOV 314, is displayed for the overlapping portion 306a (where the third FOV 316 does not also overlap the first FOV 312) and the third FOV 316 is displayed for the overlapping portion 306b. Therefore, only a portion of the first FOV 312 and only a portion of the second FOV 314 is used for the composite image 310b. The composite image 310b can include a representation of the sampling needle trajectory 110. In the third position 330, the sampling needle trajectory 110 does not intersect the nodule 302.

As the EBUS 102 is further withdrawn proximally in the airway channel 120, the EBUS 102 is moved to a fourth position 340 (see FIG. 5D) to capture a fourth FOV 304. The fourth FOV 304 is transmitted to the imaging display. In the illustrated example, the fourth FOV 304, the third FOV 316, the second FOV 314, and the first FOV 312 are combined to generate a composite image 310c. In another example, the fourth FOV 306 and the first FOV 312 are combined to generate a composite image. In the illustrated example, the first FOV 312 and the fourth FOV 304 have a nonoverlapping portion 308. In an example, as the nonoverlapping portion 308 does not include the nodule 302 nor the sampling needle trajectory 110, the composite image leaves the nonoverlapping portion 308 empty. Alternatively, the imaging display, or a computing device, generates artificial data to fill the nonoverlapping portion 308. For example, the imaging device, or the computing device interpolates data between the first FOV 312 and the fourth FOV 304 based on the landmarks present in the first FOV 312 and the fourth FOV 304. As another example, the images are combined only as distally as needed to maintain the nodule 302 (e.g., a portion of the nodule 302, a substantial portion of the nodule 302, or the entire nodule 302) within the composite image. The composite image 310c is displayed on the imaging device. In the illustrated example, the first FOV 312 and the second FOV 314 have an overlapping portion 306a, the second FOV 314 and the third FOV 316 have an overlapping portion 306b, and the third FOV 316 and the fourth FOV 304 have an overlapping portion 306c. The overlapping portions 306a, 306b, and 306c may overlap with one another. For the overlapping portions 306a, 306b, and 306c, the more recently obtained FOV is displayed for the given overlapping portion 306a, 306b, and 306c. Therefore, only a portion of the first FOV 312, only a portion of the second FOV 314, and only a portion of the third FOV 316 is used for the composite image 310c. The composite image 310c can include a representation of the sampling needle trajectory 110. In the illustrated example, the sampling needle trajectory 110 intersects the nodule 302 in the fourth position 340.

Once the sampling needle trajectory 110 intersects the nodule 302, the sampling needle is extended. As the sampling needle is extended, the fourth FOV 304 is captured and updated in real-time, such that the composite image 310c is updated in real-time. In an example, the sampling needle is extended, a graphical representation of the sampling needle is generated and displayed on the composite image 310c.

It will be appreciated that compositing or joining adjacent and potentially overlapping ultrasound images together as shown in FIGS. 5A-5D provides a marked improvement over existing ultrasound sampling systems. Specifically, compositing adjacent ultrasound images together as shown and described in relation to FIGS. 5A-5D facilitates real-time visualization of a sampling procedure being performed on a target nodule 302 that is too deep within the patient's tissue to be simultaneously within a current FOV while being intersected by the needle trajectory. For example, in embodiments in which the needle trajectory traverses the FOV from a proximal end to a distal end while diverging away from the longitudinal axis of the EBUS 102, the target nodule 302 may reside above the needle trajectory whenever the target nodule 302 is within a current FOV. By withdrawing the EBUS 102 proximally, the needle trajectory 110 may be caused to intersect with the target nodule 302. However, withdrawing the EBUS 102 as needed to cause the needle trajectory 110 to intersect the target 302 may cause the target nodule to be located distal from the distal FOV boundary. The disclosed techniques mitigate this drawback by stitching adjacent images together to effectively extend the FOV beyond the distal FOV boundary by compositing previously acquired image data onto the distal end of the current FOV.

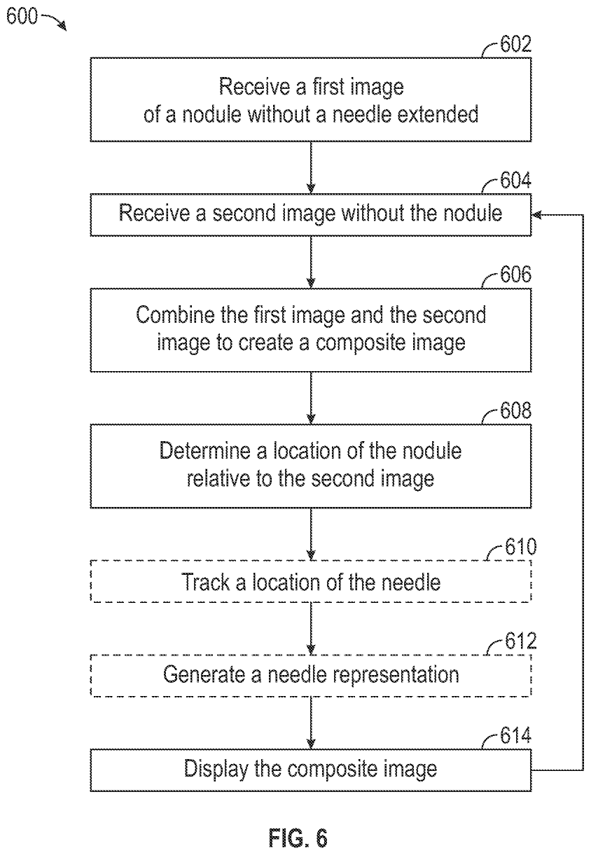

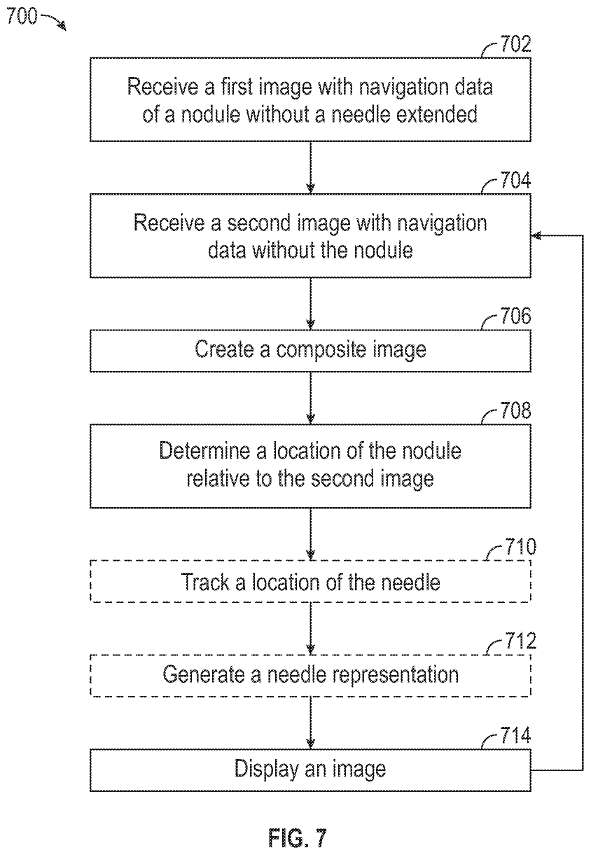

FIG. 6 illustrates an example of a method 600 of displaying a nodule when the nodule is no longer within the field of view of an EBUS without navigation in accordance with at least one example of this disclosure. Method 600 is used when a nodule (e.g., nodule 202, nodule 212, and/or nodule 302) is at a depth (e.g., second distance (D2)) in the tissue (e.g., airway tissue walls 112) that prevents the EBUS from imaging the nodule and a needle (e.g., sampling needle 108) in the same field of view (FOV) during collection of a sample of the nodule. In an example, the method 600 can be performed by a computer device (e.g., the machine of FIG. 7) communicatively coupled to the EBUS (e.g., EBUS 102 of FIGS. 1-5). The computer device includes processing circuity for conducting various image processing tasks discussed in conjunction with method 600. Further, the computer device can include an output display, such as a monitor to display the ultrasound image and various additional user interface elements, among other things.

At the beginning of method 600, such as at the beginning of a procedure, the EBUS (e.g., EBUS 102) is positioned in a first location, where the nodule is located. At 602, the computing device receives a first image of a nodule without a needle extended. The first image does not include navigation data. The nodule is located at a second distance from the EBUS, such that the nodule is within the FOV of the EBUS, but the needle and the nodule cannot be imaged simultaneously during collection of a sample from the nodule. For example, the needle and nodule cannot be imaged simultaneously during collection of a sample because of the angle that the needle exits the EBUS and the depth of the nodule do not intersect in the FOV of the EBUS. In an example, the computing device receives a plurality of first images. In an example, the computing device receives a plurality of first images at a plurality of rotational positions. For example, the computing device receives a plurality of first images at a plurality of rotational positions to determine an ideal position of the EBUS, such that the sampling needle intersects with the nodule at the largest diameter of the nodule or to the center of the nodule. Due to operation preplanning, the computing device knows the rotational room for error of the EBUS. In an example, the EBUS may be moved proximally, and therefore the computing device receives a plurality of first images that include the nodule. After the first image, or the plurality of first images, is received, the EBUS is moved proximally.

At 604, the computing device receives a second image without the nodule. The second image does not include navigation data. The computing device receives the second image in real time. Accordingly, the computing device receives a plurality of second images. In an example, the second image contains a portion of the nodule, depending on the depth of the nodule. In other words, the method 600 as describes occurs in real-time or near real-time, with the first image being a previously captured image frame (or composite of a plurality of frames), and the second image being the current real-time image (which if the EBUS is stationary could be a plurality of images coming in at the camera frame rate).

At 606, the computing device combines the first image and the second image to create a composite image. Accordingly, the composite image includes the nodule, and the current FOV (which may include a needle extended from the EBUS). In an example, the computing device combines the first image and the second image by identifying common visual landmarks between the first image and the second image. In an example, after the common visual landmarks are identified, the computing device matches the landmarks in the first image and the second image to align the first image and the second image. In an example, once the landmarks are aligned, the computing device creates the composite image. In an example, the computing device uses a portion of the first image with the second image to create the composite image. In an example, the computing device combines a plurality of first images with the second image (for example, as shown in FIGS. 5A-5D) to create the composite image. For example, the computing device uses a portion of a most distal first image to create a distal most portion of the composite image. For example, the computing device uses the most distal first image to be used to create the most distal portion of the composite image that a second most distal first image does not include. Therefore, the composite image can be formed from a plurality of previously acquired images in different distal and proximal positions. In such an example, the computing device uses the most recent image received of that portion of the patient's anatomy to create the composite image. Accordingly, computing device generates the composite image by overriding the first image (the previously acquired image) with the second image (the real-time image). Alternatively or additionally, the composite image is continually updated as the EBUS is withdrawn, such that the composite image grows linearly as additional real-time images are captured and combined with the previously acquired images. The method 600 can run at real-time and continue to build on the composite image as the EBUS device is withdrawn to properly position the sampling needle (as illustrated by the dotted line running from operation 614 back to operation 604).

At 608, the computing device determines a location of the nodule relative to the second image. In an example, the computing device determines the location of the nodule by identifying the nodule. For example, the computing device identifies the nodule by identifying an imaging signature of the nodule. As another example, the computing device identifies the nodule by receiving a user input selecting the nodule. In an example, the computing device determines the location of the nodule based on a depth of the nodule in the first image based on known field of view coordinates. In an example, the computing device determines the location of the nodule in relation to the second image based on the position of the first image in relation to the second image in the composite image and based on the known field of view coordinates.

The needle may be extended out of the exit port of the EBUS. At 610, if the needle has been extended, the computing device tracks a location of the needle. Because the distal tip of the needle will be outside the FOV when sampling the nodule, the distal tip of the needle will not be shown on the second image when the needle pierces the nodule. Therefore, the computing device tracks the location of the needle. For example, the computing device tracks the length of that the needle is extended from the EBUS. As another example, computing device tracks the trajectory of the needle as it exits the EBUS. As another example, the computing device tracks, or knows, the angle that the needle exits the EBUS. As another example, the computing device tracks the length, trajectory, and angle, or combination thereof of the needle. In an example, the computing device tracks a location of the needle by identifying the needle on the second image. In an example, the computing device tracks the location of the needle by determining the location of the needle relative to the second image. For example, the computing device identifies an imaging signature of the needle to track the location of the needle. In another example, the needle includes a visual marker to aid in tracking the location of the needle. For example, the needle includes one or more echogenic rings along a length of the needle. For example, the computing device identifies the echogenic rings, a distance between the echogenic rings, or a number of echogenic rings visible to track location of the needle. In another example, the EBUS includes a sensor at the exit port, such that the computing device receives information from the EBUS indicating a distance that the needle has been extended. From the distance information, and the known angle of exit of the needle, the computing device determines a location of the needle. In another example, the computing device tracks the location of the needle based on information received from a linear encoder in the needle actuation handle. In an example, whether or not the needle has not been extended from the exit port, the computing device predicts the needle trajectory of the needle. In an example, the computing device knows the angle that the needle exits the exit port of the EBUS and the distance between the exit port and the imaging sensor. Therefore, the computing device can determine the predicted needle trajectory without the needle being extended from the EBUS using the known angle of exit and the distance between the exit port and the imaging sensor. In an example, the computing device determines whether the predicted needle trajectory intersects with the nodule, using the location of the nodule that has been determined.

At 612, the computing device generates a needle representation. The computing device generates the needle representation based on the tracked needle location. The needle representation is a virtual, visual representation of the needle to be displayed on the imaging device. The needle representation may aid the clinician in collecting the sample from the nodule with the needle. In an example, the computing device generates a graphical representation of the extension of the needle for the needle representation. For example, the needle representation looks substantially similar to the needle to aid in identifying the needle in the image. For example, the needle representation may be a line, or a needle shaped object.

In an example, the needle representation is a representation of the predicted needle trajectory. For example, the computing device generates a graphical representation of the predicted needle trajectory. In an example, the predicted needle trajectory representation is an arrow, a line, or other representation of the predicted needle trajectory. In an example, the predicted needle trajectory representation extends to, or is located at, an intersection with the nodule. In an example, the predicted needle trajectory representation extends to, or is located at, an edge of the composite image. For example, the predicted needle trajectory representation extends to, or is located at, a top or distal edge of the composite image, depending on the angle and position of the needle. In an example, the predicted needle trajectory representation is positioned a predetermined distance from the needle representation. For example, as the computing device moves the needle representation, based on extension or retraction of the needle, the computing device moves the predicted needle trajectory representation at a fixed distance from the needle representation. In an example, the computing device generates multiple predicted needle trajectory representations. For example, the multiple predicted needle trajectory representations are positioned along the same predicted needle trajectory, but individual representations along that trajectory. In another example, the computing device determines a predicted distance between the exit port of the EBUS and the nodule. For example, the computing device uses the predicted distance to determine the length the needle should be extended from the EBUS. In an example, the computing device determines the horizontal distance between the predicted needle trajectory and the nodule. Therefore, the computing device determines how far the EBUS needs to move in order for the predicted needle trajectory, and therefore the needle, to intersect the nodule.

At 614, the computing device displays the composite image. Despite the nodule being positioned too deep into the tissue to image both the nodule and the needle in one FOV during collection of a tissue sample, the computing device generates a composite image with a modified FOV that includes the nodule and the needle. In an example, prior to the nodule leaving the FOV (e.g., of the first image) the first image is displayed in real time, unaltered by the processing described here. In an example, the computing device enables the user to switch between multiple image display modes. For example, the computing device may include a first mode that generates a composite image when a nodule is detected past a certain depth and a second mode that displays an unaltered real-time image. When a needle representation is generated, the needle representation is displayed on the composite image. As the needle continues to be extended, or retracted, the needle representation is dynamically updated in real-time on the composite image. In an example, the computing device displays a predicted distance between the exit port of the EBUS and the nodule, the predicted length the needle needs to be extended to reach the nodule, and/or the distance the EBUS needs to more for the needle to intersect the nodule. For example, displaying the predicted distance can aid with a user in determining how far to extend the needle from the EBUS. Accordingly, the computing device can aid in identifying when the nodule has been reached for sampling.

Method 600 operates on the computing device in real-time and continuously throughout the procedure. For example, while the needle is extended into the FOV of the EBUS, second images continue to be collected and method 600 operates in real-time to generate and display composite images using the second image and first image, while displaying the nodule in relation to the needle. As another example, first images will continue to be collected until a second image without the nodule is identified. As another example, first images will continue to be collected until a second image is identified in which the needle trajectory intersects the nodule. First images and second images may be continuously received and new composite images may be displayed throughout the procedure. Therefore, the computing device persistently displays imaged biopsy targets (e.g., nodules) even after the biopsy targets are no longer within the FOV of the EBUS.

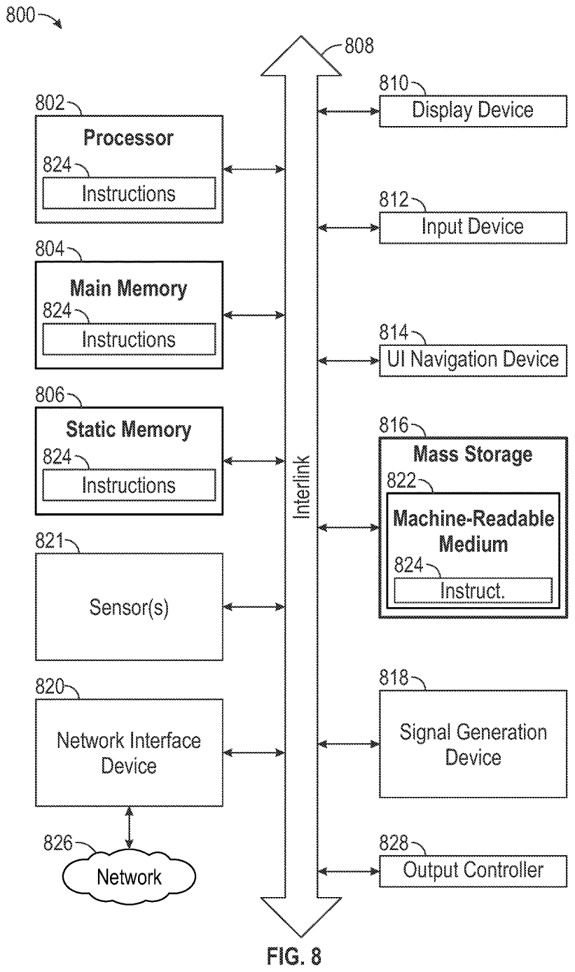

FIG. 7 illustrates an example of a method of displaying a nodule when the nodule is no longer within the field of view of an EBUS with navigation in accordance with at least one example of this disclosure. Method 700 is used when a nodule (e.g., nodule 202, nodule 212, and/or nodule 302) is at a depth (e.g., second distance (D2)) in the tissue (e.g., airway tissue walls 112) that prevents the EBUS from imaging the nodule and a needle (e.g., sampling needle 108) in the same field of view (FOV) during collection of a sample of the nodule. In an example, the method 700 can be performed by a computer device (e.g., the machine of FIG. 8) communicatively coupled to the EBUS (e.g., EBUS 102 of FIGS. 1-5). The computer device includes processing circuity for conducting various image processing tasks discussed in conjunction with method 700. Further, the computer device can include an output display, such as a monitor to display the ultrasound image and various additional user interface elements, among other things.

At the beginning of method 700, such as at the beginning of a procedure, the EBUS (e.g., EBUS 102) is positioned in a first location, where the nodule is located. EBUS devices in use today commonly include a navigation system that allows for precise tracking of position and orientation within the patient. The navigation system of the EBUS can utilize electromagnetic (EM) tracking techniques, that allow for tracking the EBUS within six degrees of freedom. In an example, the tracked positions include a position of the EBUS and an orientation of the EBUS.

At 702, the computing device receives a first image of a nodule without a needle extended. The first image includes navigation data. The nodule is located at a second distance from the EBUS, such that the nodule is within the FOV of the EBUS, but the needle and the nodule cannot be imaged simultaneously during collection of a sample from the nodule. For example, the needle and nodule cannot be imaged simultaneously during collection of a sample because of the angle that the needle exits the EBUS and the depth of the nodule do not intersect in the FOV of the EBUS. In an example, the computing device receives a plurality of first images. In an example, the computing device receives a plurality of first images at a plurality of rotational positions. For example, the computing device receives a plurality of first images at a plurality of rotational positions to determine an ideal position of the EBUS, such that the sampling needle intersects with the nodule at the largest diameter of the nodule or to the center of the nodule. Due to operation preplanning, the computing device knows the rotational room for error of the EBUS. In another example, the computing device knows the location and rotation in which the EBUS needs to be positioned to capture the nodule, such that the computing device knows if the EBUS is misaligned. In an example, the EBUS may be moved proximally, and therefore the computing device receives a plurality of first images that include the nodule. After the first image, or the plurality of first images, is received, the EBUS is moved proximally.

At 704, the computing device receives a second image without the nodule. The second image includes navigation data. The computing device receives the second image in real time. Accordingly, the computing device receives a plurality of second images. In an example, the second image contains a portion of the nodule, depending on the depth of the nodule. In other words, the method 700 as describes occurs in real-time or near real-time, with the first image being a previously captured image frame (or composite of a plurality of frames), and the second image being the current real-time image (which if the EBUS is stationary could be a plurality of images coming in at the camera frame rate).

At 706, the computing device creates a composite image. In an example, the computing device combines the first image and the second image to create the composite image. Accordingly, the composite image includes the nodule, and the current FOV (which may include a needle extended from the EBUS). The computing device combines the first image and the second image using navigation data. In an example, the computing device uses the navigation data to determine the distance between the first image and the second image. In an example, the computing device uses the distance between the first image and the second image to position the first image with respect to the second image. In an example, the computing device uses a portion of the first image with the second image to create the composite image. In an example, the computing device combines a plurality of first images with the second image (for example, as shown in FIGS. 5A-5D) to create the composite image. For example, the computing device uses a portion of a most distal first image to create a distal most portion of the composite image. For example, the computing device uses the most distal first image to be used to create the most distal portion of the composite image that a second most distal first image does not include. Therefore, the composite image can be formed from a plurality of previously acquired images in different distal and proximal positions. Alternatively or additionally, the composite image is continually updated as the EBUS is withdrawn, such that the composite image grows linearly as additional real-time images are captured and combined with the previously acquired images. In such an example, the computing device uses the most recent image received of that portion of the patient's anatomy to create the composite image. Accordingly, computing device generates the composite image by overriding a portion of the first image (the previously acquired image) with the second image (the real-time image). In another example, the computing device can interpolate data between the first image and the second image if a portion of the first image and the second image are not overlapping. For example, the computing device interpolates data between the first image and the second image by identifying common landmarks in the first image and the second image and interpolating missing portions of the common landmarks between the first image and the second image.

In another example, the computing device does not fill any space between the first image and the second image if the first image and the second image are not overlapping. For example, the first image includes the nodule and the second image includes the current FOV, which does not include the nodule. Therefore, because the clinician needs to know the locations of the nodule with respect to the current FOV to correctly position the EBUS to collect a sample of the nodule with the needle, the computing device would display the first image and the second image with a space between them based on the distance between the first image FOV and the second image FOV. In an example, the computing device displays graphical representation and other information as described below, and such representations span across the first image and the second image as appropriate, as if the data between the first image and the second image was not missing. In another example, the computing device does not create a space between the first image and the second image, but include an indication that the first image and the second image are separated by a given distance. In an example, the indication is a line or marking between the images to indicate data is missing. In another example, the computing device displays a distance between the images on the display device, and marks the edges of the images. In such an example, the computing device displays graphical representation and other information as described below, and such representations are included in the first image and the second image as appropriate, as if the data between the first image and the second image was not missing. However, the representations that would extend between the first image and the second image is not included. Therefore, certain representations, such as a predicted needle trajectory, may be offset from one another when the first image and the second image are side by side.

In another example, the computing device creates a composite image by determining the positioning between the first image and the second image. In such an example, the only data maintained from the first image may be the location information and the nodule. For example, once the location of the nodule relative to the second image is determined at 708, the composite image includes the second image and the nodule. The nodule can be positioned at the location in which the nodule would be positioned if the first image was still present. For example, the computing device creates a representation of the nodule, which is placed outside the second image, inside the second image, or partially inside the second image, depending on the location of the nodule. For example, the computing device can create a representation of the nodule that matches the shape of the nodule, is circular, is oval shaped, is an outline of the nodule, or any other graphical symbol to represent the nodule. In another example, the representation of the nodule can be a color that is easy to identify in relation to the second image.

In an example, the computing device continually updates the composite image by updating the second image in real-time as real-time second images are received.

At 708, the computing device determines a location of the nodule relative to the second image. In an example, the computing device determines the location of the nodule by identifying the nodule. For example, the computing device identifies the nodule by identifying an imaging signature of the nodule. As another example, the computing device identifies the nodule by receiving a user input selecting the nodule. In an example, the computing device determines the location of the nodule based on a depth of the nodule in the first image based on known field of view coordinates. In an example, the computing device determines the location of the nodule in relation to the second image based on the position of the first image in relation to the second image in the composite image and based on the known field of view coordinates. In another example, the computing device determines the location of the nodule by comparing the navigation data from the first image to the navigation data of the second image. For example, the navigation data from the first image indicates a first device pose and the navigation data from the second image indicates a second device pose such that the computing device can determine a relative location of the nodule in relation to the second image.

The needle is extended out of the exit port of the EBUS. At 710, if the needle has been extended, the computing device tracks a location of the needle. Because the distal tip of the needle will be outside the FOV when sampling the nodule, the distal tip of the needle will not be shown on the second image when the needle pierces the nodule. Therefore, the computing device tracks the location of the needle. For example, the computing device tracks the length of that the needle is extended from the EBUS. As another example, computing device tracks the trajectory of the needle as it exits the EBUS. As another example, the computing device tracks, or knows, the angle that the needle exits the EBUS. As another example, the computing device tracks the length, trajectory, and angle, or combination thereof of the needle. In an example, the computing device tracks a location of the needle by identifying the needle on the second image. In an example, the computing device tracks the location of the needle by determining the location of the needle relative to the second image. For example, the computing device identifies an imaging signature of the needle to track the location of the needle. In another example, the needle includes a visual marker to aid in tracking the location of the needle. For example, the needle includes one or more echogenic rings along a length of the needle. For example, the computing device identifies the echogenic rings, a distance between the echogenic rings, or a number of echogenic rings visible to track location of the needle. In another example, the EBUS includes a sensor at the exit port, such that the computing device receives information from the EBUS indicating a distance that the needle has been extended. From the distance information, and the known angle of exit of the needle, the computing device determines a location of the needle. In another example, the computing device tracks the location of the needle based on information received from a linear encoder in the needle actuation handle. In an example, the computing device tracks the location of the needle using the navigation data of the second image and/or the first image. In an example, whether or not the needle has not been extended from the exit port, the computing device predicts the needle trajectory of the needle. In an example, the computing device knows the angle that the needle exits the exit port of the EBUS and the distance between the exit port and the imaging sensor. Therefore, the computing device can determine the predicted needle trajectory without the needle being extended from the EBUS using the known angle of exit and the distance between the exit port and the imaging sensor. In an example, the computing device determines whether the predicted needle trajectory intersects with the nodule, using the location of the nodule that has been determined.

At 712, the computing device generates a needle representation. The computing device generates the needle representation based on the tracked needle location. The needle representation is a virtual, visual representation of the needle to be displayed on the imaging device. The needle representation may aid the clinician in collecting the sample from the nodule with the needle. In an example, the computing device generates a graphical representation of the extension of the needle for the needle representation. For example, the needle representation looks substantially similar to the needle to aid in identifying the needle in the image. For example, the needle representation may be a line, or a needle shaped object.

In an example, the needle representation is a representation of the predicted needle trajectory. For example, the computing device generates a graphical representation of the predicted needle trajectory. In an example, the predicted needle trajectory representation is an arrow, a line, or other representation of the predicted needle trajectory. In an example, the predicted needle trajectory representation extends to, or is located at, an intersection with the nodule. In an example, the predicted needle trajectory representation extends to, or is located at, an edge of the composite image. For example, the predicted needle trajectory representation extends to, or is located at, a top or distal edge of the composite image, depending on the angle and position of the needle. In an example, the predicted needle trajectory representation is positioned a predetermined distance from the needle representation. For example, as the computing device moves the needle representation, based on extension or retraction of the needle, the computing device moves the predicted needle trajectory representation at a fixed distance from the needle representation. In an example, the computing device generates multiple predicted needle trajectory representations. For example, the multiple predicted needle trajectory representations are positioned along the same predicted needle trajectory, but individual representations along that trajectory. In another example, the computing device determines a predicted distance between the exit port of the EBUS and the nodule. For example, the computing device uses the predicted distance to determine the length the needle should be extended from the EBUS. In an example, the computing device determines the horizontal distance between the predicted needle trajectory and the nodule. Therefore, the computing device determines how far the EBUS needs to move in order for the predicted needle trajectory, and therefore the needle, to intersect the nodule.

At 714, the computing device displays an image. Despite the nodule being positioned too deep into the tissue to image both the nodule and the needle in one FOV during collection of a tissue sample, the computing device generates a composite image with a modified FOV that includes the nodule and the needle. For example, computing device display the composite image. In an example, prior to the nodule leaving the FOV (e.g., of the first image) the first image is displayed in real time, unaltered by the processing described here. In an example, the computing device enables the user to switch between multiple image display modes. For example, the computing device may include a first mode that generates a composite image when a nodule is detected past a certain depth and a second mode that displays an unaltered real-time image. In an example, the computing device displays an image including the second image and a marking indicating the location of the nodule. For example, the computing device places the marking indicating the location of the nodule outside the second image. For example, the computing device creates a marking that is shaped substantially similar to the nodule, a round marking, an oval-shaped marking, or other shape to indicate the nodule. When a needle representation is generated, the computing device displays the needle representation on the image. As the needle continues to be extended, or retracted, the needle representation is dynamically updated in real-time on the composite image. In an example, the computing device displays a predicted distance between the exit port of the EBUS and the nodule, the predicted length the needle needs to be extended to reach the nodule, and/or the distance the EBUS needs to more for the needle to intersect the nodule. For example, displaying the predicted distance can aid with a user in determining how far to extend the needle from the EBUS. Accordingly, the computing device can aid in identifying when the nodule has been reached for sampling.

Method 700 operates on the computing device in real-time and continuously throughout the procedure. For example, while the needle is extended into the FOV of the EBUS, second images continue to be collected and method 700 operates in real-time to generate and display composite images using the second image and first image, while displaying the nodule in relation to the needle. As another example, first images will continue to be collected until a second image without the nodule is identified. As another example, first images will continue to be collected until a second image is identified in which the needle trajectory intersects the nodule. First images and second images may be continuously received and new composite images may be displayed throughout the procedure. Therefore, the computing device persistently displays imaged biopsy targets (e.g., nodules) even after the biopsy targets are no longer within the FOV of the EBUS.

The steps or operations of the methods 600 and 700 are illustrated in a particular order for convenience and clarity; many of the discussed operations can be performed in a different sequence or in parallel without materially impacting other operations. The methods 600 and 700 as discussed includes operations performed by multiple different actors, devices, and/or systems. It is understood that subsets of the operations discussed in the methods 600 and 700 can be attributable to a single actor, device, or system could be considered a separate standalone process or method.

The above invention is discussed in reference to an endoscope application, but the same concept can be applied to endoscopic, ultrasound, and microscope diagnostic applications. The system described herein can transition between AI modules intra-procedurally and will attach metadata that identifies which AI modules is operating for different portions of a video stream. The system supports modular insertion of AI modules as functionality is added or updated. The system can generate reports detailing which algorithms (modules) where selected and why throughout a procedure.

FIG. 8 illustrates a block diagram of an example machine 800 upon which any one or more of the techniques (processes) discussed herein may perform in accordance with some embodiments. In alternative embodiments, the machine 800 may operate as a standalone device and/or may be connected (e.g., networked) to other machines. In a networked deployment, the machine 800 may operate in the capacity of a server machine, a client machine, or both in server-client network environments. In an example, the machine 800 may act as a peer machine in peer-to-peer (P2P) (or other distributed) network environment. The machine 800 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 800 may include a hardware processor 802 (e.g., a central processing unit (CPU), a graphics processing unit (GPU), a hardware processor core, or any combination thereof), a main memory 804 and a static memory 806, some or all of which may communicate with each other via an interlink (e.g., bus) 808. The machine 800 may further include a display unit 810, an alphanumeric input device 812 (e.g., a keyboard), and a user interface (UI) navigation device 814 (e.g., a mouse). In an example, the display unit 810, input device 812 and UI navigation device 814 may be a touch screen display. The machine 800 may additionally include a storage device (e.g., drive unit) 816, a signal generation device 818 (e.g., a speaker), a network interface device 820, and one or more sensors 821, such as a global positioning system (GPS) sensor, compass, accelerometer, or other sensor. The machine 800 may include an output controller 828, such as a serial (e.g., Universal Serial Bus (USB), parallel, or other wired or wireless (e.g., infrared (IR), near field communication (NFC), etc.) connection to communicate and/or control one or more peripheral devices (e.g., a printer, card reader, etc.).

The storage device 816 may include a machine readable medium 822 on which is stored one or more sets of data structures or instructions 824 (e.g., software) embodying or utilized by any one or more of the techniques or functions described herein. The instructions 824 may also reside, completely or at least partially, within the main memory 804, within static memory 806, or within the hardware processor 802 during execution thereof by the machine 800. In an example, one or any combination of the hardware processor 802, the main memory 804, the static memory 806, or the storage device 816 may constitute machine readable media.

While the machine readable medium 822 is illustrated as a single medium, the term “machine readable medium” may include a single medium or multiple media (e.g., a centralized or distributed database, and/or associated caches and servers) configured to store the one or more instructions 824. The term “machine readable medium” may include any medium that is capable of storing, encoding, or carrying instructions for execution by the machine 800 and that cause the machine 800 to perform any one or more of the techniques of the present disclosure, or that is capable of storing, encoding or carrying data structures used by or associated with such instructions. Non-limiting machine-readable medium examples may include solid-state memories, and optical and magnetic media.

The instructions 824 may further be transmitted or received over a communications network 826 using a transmission medium via the network interface device 820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 820 may include one or more physical jacks (e.g., Ethernet, coaxial, or phone jacks) or one or more antennas to connect to the communications network 826. In an example, the network interface device 820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 800, and includes digital or analog communications signals or other intangible medium to facilitate communication of such software.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

### EXAMPLES

The following examples provide a non-limiting array of examples of the present disclosure.

Example 1 is a method for real-time combination of an ultrasound imaging stream. The method comprises receiving, via the ultrasound imaging stream, at least one first image of a first field of view of a patient, wherein the first field of view includes a nodule; receiving, via the ultrasound imaging stream, a plurality of second images of a second field of view of the patient, the plurality of second images received subsequent to the at least one first image, wherein the second field of view is proximal the first field of view and wherein the second field of view does not include the nodule; generating a plurality of composite images by combing one of the plurality of second images with at least a portion of the at least one first image, the at least the portion of the at least one first image including the nodule; and displaying the plurality of composite images in substantially real-time as the receiving the plurality of second images. The the context of these examples “in substantially real-time” is intended to mean as quickly as the processing circuitry can produce the composite images.

In Example 2, the subject matter of Example 1 optionally includes determining a location of the nodule relative to at least one of the plurality of second images based on an image signature of the nodule.

In Example 3, the subject matter of Examples 1 or 2 optionally includes determining a location of the nodule relative to at least one of the plurality of second images based on user input, received via a user device, indicating the nodule.

In Example 4, the subject matter of any one of Examples 1 to 3 optionally includes determining a location of the nodule relative to at least one of the plurality of second images by determining a depth of the nodule in the at least one first image based on known field of view coordinates, wherein the depth of the nodule is such that a trajectory of the needle does not intersect the nodule in the first field of view.

In Example 5, the subject matter of any one of Examples 1 to 4 optionally includes determining a location of the nodule relative to at least one of the plurality of second images by determining the location of the nodule in relation to the one of the plurality of second image based on the position of the at least one first image in relation to one of the plurality of second images in the composite image and based on known field of view coordinates.

In Example 6, the subject matter of any one of Examples 1 to 5 optionally includes determining a location of the nodule relative to at least one of the plurality of second images by comparing a set of navigation data of the at least one first image to a set of navigation data of one of the plurality of second images.

In Example 7, the subject matter of any one of Examples 1 to 6 optionally includes generating a plurality of composite images by combing one of the plurality of second images with at least a portion of the at least one first image comprises positioning the at least one first image and one of the plurality of second images by identifying and aligning landmarks similar between the at least one first image and one of the plurality of second images.

In Example 8, the subject matter of any one of Examples 1 to 6 optionally includes generating a plurality of composite images by combing one of the plurality of second images with at least a portion of the at least one first image comprises positioning the at least one first image and one of the plurality of second images by determining a distance between the at least one first image and one of the plurality of second images using a set of navigation data of the at least one first image and a set of navigation data of one of the plurality of second images.

In Example 9, the subject matter of any one of Examples 1 to 8 optionally includes identifying that a needle has been extended in the second field of view of one of the plurality of second images.

In Example 10, the subject matter of any one of Examples 1 to 9 optionally includes generating a needle representation.

In Example 11, the subject matter of any one of Examples 1 to 10 optionally includes where the needle representation indicates a length that the needle is extended.

In Example 12, the subject matter of any one of Examples 1 to 11 optionally includes displaying the plurality of composite images comprises displaying the needle representation on the plurality of composite images.

In Example 13, the subject matter of any one of Examples 1 to 12 optionally includes generating a predicted needle trajectory representation.

In Example 14, the subject matter of any one of Examples 1 to 13 optionally includes displaying the plurality of composite images comprises displaying the predicted needle trajectory representation on the plurality of composite images.

In Example 15, the subject matter of any one of Examples 1 to 14 optionally includes displaying the predicted needle trajectory representation on the plurality of composite images comprises displaying the predicted needle trajectory representation intersecting the nodule.

In Example 16, the subject matter of any one of Examples 1 to 15 optionally includes generating a plurality of composite images by combing one of the plurality of second images with at least a portion of the at least one first image comprises updating one of the plurality of second images in real-time.

Example 17 is a method for real-time combination of an ultrasound imaging stream. The method comprises receiving, via the ultrasound imaging stream, at least one first image of a first field of view of a patient, wherein the first field of view includes a nodule; receiving, via the ultrasound imaging stream, at least one second image of a second field of view of the patient, wherein the second field of view is proximal the first field of view; receiving, via the ultrasound imaging stream, a plurality of third images of a third field of view of the patient, the plurality of third images received subsequent to the at least one first image and the at least one second image, wherein the third field of view is proximal the second field of view; generating a plurality of composite images by combing the plurality of third images with at least a portion of the at least one first image and at least a portion of the at least one second image; and displaying the plurality of composite images in substantially real-time as the receiving the plurality of third images.

In Example 18, the subject matter of Example 17 optionally includes determining a location of the nodule relative to one of the plurality of third images.

In Example 19, the subject matter of any one of Examples 17 or 18 optionally includes identifying that a needle has been extended in the third field of view of one of the plurality of third images.

In Example 20, the subject matter of any one of Examples 17 to 19 optionally includes generating a predicted needle trajectory representation and wherein displaying the plurality of composite images comprises displaying the needle representation on the plurality of composite images.

Example 21 is an internal medical imaging system. The system comprises an imaging sensor configured to obtain medical images within an internal portion of a patient; a display device configured to display image data and related graphical information from the imaging sensor; and a computing device including a processor and a memory device, the memory device including instructions that, when executed by the processor, cause the computing device to perform operations including: receive, from the imaging sensor, at least one first image of a first field of view of a patient, wherein the first field of view includes a nodule; receive, from the imaging sensor, a plurality of second images of a second field of view of the patient, the plurality of second images received subsequent to the at least one first image, where the second field of view is proximal the first field of view; generate a plurality of composite images by combing one of the plurality of second images with at least a portion of the at least one first image; generate a plurality of composite images by combing one of the plurality of second images with at least a portion of the at least one first image; and display, via the display device, the plurality of composite images in substantially real-time as the receiving the plurality of second images. The system of Example 21 can optionally perform any of the methods of Examples 1 to 20.

In Example 22, the subject matter of Example 21 optionally includes determining a location of the nodule relative to one of the plurality of second images.

In Example 23, the subject matter of any one of Examples 21 or 22 optionally includes identifying that a needle has been extended in the second field of view of one of the plurality of second images.

In Example 24, the subject matter of any one of Examples 21 to 23 optionally includes receiving, from the imaging sensor, at least one third image of a third field of view of the patient, wherein the third field of view is between the first field of view and the second field of view; and wherein generating the plurality of composite images comprises combing the plurality of third images with at least a portion of the at least one first image and at least a portion of the at least one second image.

In Example 25, the subject matter of any one of Examples 21 to 24 optionally includes generating a predicted needle trajectory representation, and wherein displaying the plurality of composite images comprises displaying the needle representation on the plurality of composite images.

Example 26 is a machine-readable medium, storing instructions, which when executed by a machine, cause the machine to perform operations comprising: receiving, via the ultrasound imaging stream, at least one first image of a first field of view of a patient, wherein the first field of view includes a nodule; receiving, via the ultrasound imaging stream, a plurality of second images of a second field of view of the patient, the plurality of second images received subsequent to the at least one first image, wherein the second field of view is proximal the first field of view and wherein the second field of view does not include the nodule; generating a plurality of composite images by combing one of the plurality of second images with at least a portion of the at least one first image, the at least the portion of the at least one first image including the nodule; and displaying the plurality of composite images in substantially real-time as the receiving the plurality of second images.

In Example 27, the subject matter of Example 26 optionally includes instructions for determining a location of the nodule relative to at least one of the plurality of second images based on an image signature of the nodule.

In Example 28, the subject matter of Example 26 optionally also includes instructions for determining a location of the nodule relative to at least one of the plurality of second images based on user input, received via a user device, indicating the nodule.

In Example 29, the subject matter of Example 26 optionally also includes instructions for determining a location of the nodule relative to at least one of the plurality of second images by determining a depth of the nodule in the at least one first image based on known field of view coordinates, wherein the depth of the nodule is such that a trajectory of the needle does not intersect the nodule in the first field of view.

In Example 30, the subject matter of Example 26 optionally also includes instructions for determining a location of the nodule relative to at least one of the plurality of second images by determining the location of the nodule in relation to the one of the plurality of second image based on the position of the at least one first image in relation to one of the plurality of second images in the composite image and based on known field of view coordinates.

In Example 31, the subject matter of Example 26 optionally also includes instructions for determining a location of the nodule relative to at least one of the plurality of second images by comparing a set of navigation data of the at least one first image to a set of navigation data of one of the plurality of second images.

In Example 32, the subject matter of Example 26 optionally also includes instructions for generating a plurality of composite images by combing one of the plurality of second images with at least a portion of the at least one first image comprises positioning the at least one first image and one of the plurality of second images by identifying and aligning landmarks similar between the at least one first image and one of the plurality of second images.

In Example 33, the subject matter of Example 26 optionally also includes instructions for generating a plurality of composite images by combing one of the plurality of second images with at least a portion of the at least one first image comprises positioning the at least one first image and one of the plurality of second images by determining a distance between the at least one first image and one of the plurality of second images using a set of navigation data of the at least one first image and a set of navigation data of one of the plurality of second images.

In Example 34, the subject matter of Example 26 optionally also includes instructions for identifying that a needle has been extended in the second field of view of one of the plurality of second images.

In Example 35, the subject matter of any one of Examples 26 to 34 optionally includes instructions for generating a needle representation.

In Example 36, the subject matter of any one of Examples 26 to 35 optionally includes instructions where the needle representation indicates a length that the needle is extended.

In Example 37, the subject matter of any one of Examples 26 to 36 optionally includes instructions for displaying the plurality of composite images comprises displaying the needle representation on the plurality of composite images.

In Example 38, the subject matter of any one of Examples 26 to 37 optionally includes instructions for generating a predicted needle trajectory representation.

In Example 39, the subject matter of any one of Examples 26 to 38 optionally includes instructions for displaying the plurality of composite images comprises displaying the predicted needle trajectory representation on the plurality of composite images.

In Example 40, the subject matter of any one of Examples 26 to 39 optionally includes instructions for displaying the predicted needle trajectory representation on the plurality of composite images comprises displaying the predicted needle trajectory representation intersecting the nodule.

In Example 41, the subject matter of any one of Examples 26 to 40 optionally includes instructions for generating a plurality of composite images by combing one of the plurality of second images with at least a portion of the at least one first image comprises updating one of the plurality of second images in real-time.

Example 42 is a machine-readable medium, storing instructions, which when executed by a machine, cause the machine to perform operations comprising receiving, via the ultrasound imaging stream, at least one first image of a first field of view of a patient, wherein the first field of view includes a nodule; receiving, via the ultrasound imaging stream, at least one second image of a second field of view of the patient, wherein the second field of view is proximal the first field of view; receiving, via the ultrasound imaging stream, a plurality of third images of a third field of view of the patient, the plurality of third images received subsequent to the at least one first image and the at least one second image, wherein the third field of view is proximal the second field of view; generating a plurality of composite images by combing the plurality of third images with at least a portion of the at least one first image and at least a portion of the at least one second image; and displaying the plurality of composite images in substantially real-time as the receiving the plurality of third images.

In Example 43, the subject matter of Example 42 includes instructions for determining a location of the nodule relative to one of the plurality of third images.

In Example 44, the subject matter of Example 42 includes instructions for identifying that a needle has been extended in the third field of view of one of the plurality of third images.

In Example 45, the subject matter of Examples 42 and 44 includes instructions for generating a predicted needle trajectory representation and wherein displaying the plurality of composite images comprises displaying the needle representation on the plurality of composite images.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1-25. (canceled)

26. A method for real-time combination of an ultrasound imaging stream, the method comprising:
receiving, via the ultrasound imaging stream, at least one first image of a first field of view of a patient, wherein the first field of view includes a nodule;
receiving, via the ultrasound imaging stream, a plurality of second images of a second field of view of the patient, the plurality of second images received subsequent to the at least one first image, wherein the second field of view is proximal the first field of view and wherein the second field of view does not include the nodule;
generating a plurality of composite images by combing one of the plurality of second images with at least a portion of the at least one first image, the at least the portion of the at least one first image including the nodule, wherein the plurality of composite images includes a predicted needle trajectory representation; and
displaying the plurality of composite images in substantially real-time as the receiving the plurality of second images.

27. The method of claim 26, further comprising determining a location of the nodule relative to at least one of the plurality of second images based on an image signature of the nodule.

28. The method of claim 26, further comprising determining a location of the nodule relative to at least one of the plurality of second images based on user input, received via a user device, indicating the nodule.

29. The method of claim 26, further comprising determining a location of the nodule relative to at least one of the plurality of second images by determining a depth of the nodule in the at least one first image based on known field of view coordinates, wherein the depth of the nodule is such that a trajectory of the needle does not intersect the nodule in the first field of view.

30. The method of claim 26, further comprising determining a location of the nodule relative to at least one of the plurality of second images by determining the location of the nodule in relation to the one of the plurality of second image based on a position of the at least one first image in relation to one of the plurality of second images in the composite image and based on known field of view coordinates.

31. The method of claim 26, further comprising determining a location of the nodule relative to at least one of the plurality of second images by comparing a set of navigation data of the at least one first image to a set of navigation data of one of the plurality of second images.

32. The method of claim 26, wherein generating a plurality of composite images by combing one of the plurality of second images with at least a portion of the at least one first image comprises positioning the at least one first image and one of the plurality of second images by identifying and aligning landmarks similar between the at least one first image and one of the plurality of second images.

33. The method of claim 26, wherein generating a plurality of composite images by combing one of the plurality of second images with at least a portion of the at least one first image comprises positioning the at least one first image and one of the plurality of second images by determining a distance between the at least one first image and one of the plurality of second images using a set of navigation data of the at least one first image and a set of navigation data of one of the plurality of second images.

34. The method of claim 26, further comprising identifying that a needle has been extended in the second field of view of one of the plurality of second images.

35. The method of claim 34, further comprising generating a needle representation.

36. The method of claim 35, where the needle representation indicates a length that the needle is extended.

37. The method of claim 35, wherein displaying the plurality of composite images comprises displaying the needle representation on the plurality of composite images.

38. The method of claim 35, wherein the predicted needle trajectory representation is aligned with the needle representation.

39. The method of claim 26, wherein displaying the predicted needle trajectory representation on the plurality of composite images comprises displaying the predicted needle trajectory representation intersecting the nodule.

40. The method of claim 26, wherein generating a plurality of composite images by combing one of the plurality of second images with at least a portion of the at least one first image comprises updating one of the plurality of second images in real-time.

41. An internal medical imaging system comprising:
an imaging sensor configured to obtain medical images within an internal portion of a patient;
a display device configured to display image data and related graphical information from the imaging sensor; and
a computing device including a processor and a memory device, the memory device including instructions that, when executed by the processor, cause the computing device to perform operations including:
receive, from the imaging sensor, at least one first image of a first field of view of a patient, wherein the first field of view includes a nodule;
receive, from the imaging sensor, a plurality of second images of a second field of view of the patient, the plurality of second images received subsequent to the at least one first image, where the second field of view is proximal the first field of view;
generate a plurality of composite images by combing one of the plurality of second images with at least a portion of the at least one first image;
generate a plurality of composite images by combing one of the plurality of second images with at least a portion of the at least one first image, wherein the plurality of composite images includes a predicted needle trajectory representation across at least a portion of the plurality of composite images; and
display, via the display device, the plurality of composite images in substantially real-time as the receiving the plurality of second images.

42. The internal medical imaging system of claim 41, wherein the instructions further cause the computing device to perform operations including:
determine a location of the nodule relative to one of the plurality of second images.

43. The internal medical imaging system of claim 41, wherein the instruction further cause the computing device to perform operations including:
identify that a needle has been extended in the second field of view of one of the plurality of second images.

44. The internal medical imaging system of claim 41, wherein the instructions further cause the computing device to perform operations including:
receive, from the imaging sensor, at least one third image of a third field of view of the patient, wherein the third field of view is between the first field of view and the second field of view; and
wherein generating the plurality of composite images comprises combing the plurality of third images with at least a portion of the at least one first image and at least a portion of the at least one second image.

45. The internal medical imaging system of claim 41, wherein the instructions further cause the computing device to perform operations including:
generating the predicted needle trajectory representation intersecting with the nodule.
